(19) United States
(12) Patent Application Publication
(10) Pub. No.: US 2007/0265675 A1
(43) Pub. Date: Nov. 15, 2007

(54) Testing Efficacy of Therapeutic Mechanical or Electrical Nerve or Muscle Stimulation

(75) Inventors: Robert E. Lund, St. Michael, MN (US); Karen Pilney Montpetit, Mendota Heights, MN (US); John Jason Buysman, Minnetonka, MN (US); Guangjian Wang, Falcon Heights, MN (US); John F. Otte, St. Anthony, MN (US)

Correspondence Address: AMS RESEARCH CORPORATION, 10700 BREN ROAD WEST, MINNETONKA, MN, 55343, US

(73) Assignee: AMS RESEARCH CORPORATION, Minnetonka, MN (US)

(21) Appl. No.: 11/746,476

(22) Filed: May 9, 2007

Related U.S. Application Data
(60) Provisional application No. 60/746,838, filed on May 9, 2006.

(51) Int. Cl. A61N 1/32

(52) U.S. Cl. 607/41; 607/39

(57) ABSTRACT

Methods and apparatus for testing of the efficacy of therapeutic stimulation of pelvic nerves or musculature to alleviate one of incontinence or sexual dysfunction are disclosed. A therapy delivery device is operable in a therapy delivery mode and a test mode and an evoked response detector is employed in the test mode to detect the evoked response to applied test stimuli. The test stimuli parameters of the test stimulation regimen are adjusted prior to delivery of each test stimulation regimen, and the evoked responses to the applied test stimulation regimens are compared to ascertain an optimal test stimulation regimen. The therapy stimulation regimen parameters are selected as a function of the test electrical stimulation parameters causing the optimal evoked response.

### RELATED APPLICATIONS

This application claims priority to U.S. Provisional Application Ser. No. 60/746,838 filed May, 9, 2006, the entire content of which is incorporated herein by reference.

### TECHNICAL FIELD

The present invention pertains to methods and apparatus for positioning a mechanical body stimulator or a stimulation electrode and testing the efficacy of therapeutic mechanical or electrical nerve or sphincter muscle stimulation, respectively.

### BACKGROUND

Incontinence

As set forth in U.S. Pat. No. 6,964,643, urinary incontinence is a significant clinical problem and a major source of disability and dependency. The most frequently occurring types of urinary incontinence are stress incontinence, urge incontinence, overflow incontinence, and mixed incontinence.

Stress incontinence is a common form of incontinence in women. Intraabdominal pressure exceeds urethral pressure upon coughing, sneezing, laughing, lifting, or like activity, causing leakage of urine. Physical changes associated with pregnancy, childbirth, and menopause, for example, are known to cause stress incontinence.

Urge incontinence occurs when a patient loses urine while suddenly feeling the urge to urinate. The patient is unable to inhibit the flow of urine long enough to reach the toilet. Inappropriate bladder contractions are the most common cause of urge incontinence, and may occur in connection with central nervous system lesions, urinary infection, or bladder tumors, to name several examples.

Overflow incontinence occurs when the bladder is unable to empty normally. Weak bladder muscles, caused e.g. by nerve damage from diabetes, or a blocked urethra, caused e.g. by tumors or urinary stones, are among the more common causes of overflow incontinence. Frequency or urgency involves the need or urge to urinate on an excessively frequent or habitual basis. Combinations of these and other types of incontinence, e.g. stress incontinence and urge incontinence, are often called mixed incontinence.

Many options are available to treat incontinence in its various forms, including Kegel exercises, electrical stimulation, biofeedback, timed voiding or bladder training, medications, pessaries, implantation of urethral slings, invasive or minimally invasive surgery, catheterization, and other methods and devices.

Sexual Dysfunction

Sexual dysfunction of the penis is a common problem afflicting males of all ages, genders, and races. Erectile dysfunction is a serious condition for many men, and it may include a variety of problems. Some of these problems include the inability to create an erection, incomplete erections and brief erectile periods. These conditions may be associated with nervous system disorders, and may be caused by aging, injury, or illness.

In some cases, erectile dysfunction can be attributed to improper nerve activity that incompletely stimulates the penis. For example, stimulation from the brain during arousal and sexual activity is responsible for activating an erection. With respect to erectile disorders, the problem may be a lack of sufficient stimulation from the brain, or a break in communication of the stimulation. Erectile disorders may additionally or alternatively involve dysfunctional parasympathetic function that can be attributed to many factors including illness or injury.

Methods for treating erectile dysfunction include pharmaceutical treatment and electrical stimulation. Delivery of electrical stimulation to nerves running through the pelvic floor may provide an effective therapy for many patients. For example, an implantable stimulator may be provided to deliver electrical stimulation to the pudendal or cavernous nerves to induce an erection.

Electrical Stimulation

According to several known surgical treatment methods to treat incontinence or sexual dysfunction, a neurostimulator or neuromodulator implantable medical device (IMD) is implanted in a patient's body to electrically stimulate nerves controlling external sphincter and bladder functions, e.g., the sacral nerves in the nerve root or at the peripheral sciatic nerve or the pudendal nerve to restore sexual function. One or more nerve stimulation electrode supported at the distal end of a neural lead is disposed at a nerve stimulation site, and the proximal lead connector is coupled to a connector header of an implantable pulse generator (IPG) so that the IPG and neural lead comprise the IMD. See for example, U.S. Pat. Nos. 5,569,351, 4,607,639, 4,739,764, 4,771,779, and 6,055,456, and U.S. Patent Application Publication No. 2006/0004429, regarding electrical stimulation to control bladder function.

In the process of implanting an IMD for stimulating the sacral nerves to treat incontinence, it is necessary to test the efficacy of the applied stimulation after the neural electrodes are placed at the stimulation site. In one approach, a pathway for passing the neural lead is created by a needle passed through the skin over the sacrum, through underlying tissue, and through a sacral foramen to dispose the needle tip near the sacral nerve. Electrical stimulation is applied to the needle shaft external to the skin incision, and the stimulation is conducted through the needle shaft to the stimulation site at the needle tip. As electrical stimuli are applied, the patient is asked to report any physical sensation. A relatively strong sensation is felt in the pelvic region when the sacral nerve responds to the electrical stimuli. The stimuli parameters are adjusted during this testing to attempt to determine the lowest energy stimuli that the patient can feel in the pelvic region.

This subjective testing provides an approximate confirmation that at least a certain level of stimulation evokes a response, but it does not necessarily confirm that the stimulation will effectively provide incontinence relief. In subsequent steps, a permanent or temporary neural lead is placed through the pathway, and electrical stimuli are applied through the neural lead electrodes, and testing is repeated with the patient reporting physical sensations.

In certain methods, e.g., as described in U.S. Pat. No. 6,104,960 temporary neural stimulation leads are implanted in this manner to dispose the lead electrode(s) near the sacral nerve, tunneled under the skin, extended percutaneously through the skin and coupled directly or through a cable to a patient-worn, external neural stimulator. The external neural stimulator provides stimulation for a period of days or weeks to determine if a delivered stimulation regimen is efficacious. In some cases, the patient is allowed to alter stimulation parameters and record daily urge events during this test phase. The results are evaluated as they are conducted and at the end of the test period. In some cased, it is necessary to reposition the neural electrodes and repeat the test phase. If a selected stimulation proves efficacious, the temporary neural lead is explanted, and a permanent neural lead is implanted in the pathway and coupled to an IPG that is then subcutaneously implanted.

Due to the duration and the temporary nature of the test phase, there is a high incidence of wound exposure and possibility of infection and cosmetic blemishes at the extension cable site and along the tunneling path. In addition, due to lead placement adjustments, there is also a risk of infection due to reimplanting or repositioning the neural lead. It would be desirable to avoid these complications and eliminate the lengthy and expensive test phase.

Stimulation of the pudendal nerve employing a neurostimulator IMD as an alternative to sacral nerve stimulation has long been proposed. Electrical stimulation delivered by an intravaginal or a perineal surface electrode has been shown to inhibit premature and inappropriate detrusor contractions. The mechanism for such effects appears to derive from the electrical stimulation of pudendal nerve afferents (sensory receptors or sensory nerve fibers). Input into the pudendal afferent system inhibits a parasympathetic reflex loop consisting of bladder wall afferents (sensory reflexes) and efferents (motor reflexes). This parasympathetic loop normally senses a distension of the bladder via the afferent limb and responds by sending an efferent signal to contract the bladder. Although such stimulation has shown therapeutic effects, electrode placement and on-going stimulation do not lend themselves easily to chronic stimulation.

In another approach, a muscle tissue stimulator IMD is implanted in a patient's body to directly electrically excitable muscle tissue of a sphincter, e.g., tissue structure around the urethra. For convenience, the expressions “tissue stimulator” and “tissue stimulation” may be employed herein to characterize IMDs comprising IPGs and medical electrical leads that generate and apply stimulation to tissue structures of the abdominopelvic or simply pelvic region to enervate to cause muscle tissues to contract. Exemplary muscle tissue stimulator IMDs (or simply tissue stimulators) for treatment of urinary incontinence and neurogenic bladder dysfunction are disclosed, for example, in Biocontrol Medical Ltd. U.S. Pat. Nos. 6,354,991, 6,652,449, 6,712,772, and 6,862,480 and U.S. Patent Application Publication 2005/0216069. The tissue stimulators disclosed in the Biocontrol Medical patents for treatment of both urinary stress incontinence and urge incontinence comprise a control unit or IPG and one or more medical electrical leads bearing one or more sensing/stimulation electrode and one or more physiologic sensor adapted to be implanted in selected sites of a patient's body. The sensing/stimulation electrode(s) is preferably implanted in the pelvic region of a patient so as to be in electrical contact with body tissue including one or more of the muscles that relax and contract in regulating urine flow from the bladder. The control unit is preferably implanted under the skin of the abdomen or genital region, and receives signals from the electrodes and/or from the sensors. Motion and/or pressure signals detected by the physiologic sensor(s) and/or electromyogram (EMG) signals appearing across the sensing/stimulation electrodes are conveyed to and analyzed by the control unit operating system in order to distinguish between signals indicative of urge incontinence and those indicative of stress incontinence. A particular pressure sensor design is disclosed in the above-referenced '772 patent. When impending stress incontinence is detected, the control unit generates and provides an electrical stimulation therapy having stimulation parameters configured to treat stress incontinence through the electrodes to the tissue. Similarly, urge incontinence is treated with intermittent electrical stimulation having stimulation parameters configured to treat urge incontinence.

In various configurations, the tissue stimulators disclosed in the above-referenced Biocontrol Medical patents may be used alternatively or additionally to treat fecal incontinence, interstitial cystitis, urine retention, or other sources of pelvic dysfunction, pain or discomfort, by suitable modifications to the IMD.

The control unit or IPG disclosed in the above-referenced Biocontrol Medical patents is preferably implanted under the skin of the abdomen or genital region, the stimulation/sense electrodes are preferably implanted in the pelvic region so as to be in electrical contact with one or more of the muscles that regulate urine flow from the bladder, e.g., the urethral sphincter and the levator ani, and the mechanical sensors are preferably implanted on, in or in the vicinity of the bladder. The stimulation/sense electrodes are described as flexible wire, intramuscular-type, electrodes, about 1-5 mm long and 50-100 microns in diameter, and may be formed in the shape of a spiral or hook, so that the shape facilitates fixation in tissue. The mechanical sensors supported on a sensor lead comprise one or more pressure, force, motion or acceleration sensor, or an ultrasound transducer, that generate signals responsive to motion, to intravesical or abdominal pressure, or to urine volume in the bladder, and are thus indicative of possible imminent incontinence.

Sensing circuitry in the control unit or IPG receives and processes electromyographic signals or the electromyogram (EMG) sensed across the electrodes and the mechanical sensor output signal to distinguish between EMG signals indicative of urge incontinence, EMG signals indicative of stress incontinence, and EMG signals that are not due to incontinence. Electrical stimulation pulses having stimulation parameters tailored to inhibit urge incontinence are generated by the IPG and delivered across the electrodes when the sensed signals are indicative of impending urge incontinence. Similarly, electrical stimulation pulses having stimulation parameters tailored to inhibit stress incontinence are generated by the IPG and delivered across the electrodes when the sensed signals are indicative of impending stress incontinence.

Mechanical Nerve Stimulation

Although treatments requiring surgical intervention may be the preferred and most effective treatment mode in some situations, surgical intervention may be too extreme a measure in other situations. In some cases, surgical procedures to treat incontinence actually have a relatively low success rate; in many cases such procedures are irreversible. Additionally, a patient may hesitate to proceed with a surgical option, and/or a patient's physical condition may make surgical intervention inappropriate. Surgery may be inappropriate for pregnant patients, for example, or those of advanced age. Similarly, pharmacological treatment options may cause undesirable side effects and/or interactions with other medications. Non-surgical treatments, for example exercises or bladder training, may demand too high a degree of patient compliance or effort and thus may be resisted or otherwise ineffective.

One non-surgical option that has been clinically implemented involves mechanically stimulating the patient's sacral and/or pudendal nerve as described in the above-referenced '643 patent. The periodic treatments disclosed in the '643 patent are designed to cause certain nerve responses or otherwise minimize urinary and/or fecal incontinence in one or more of the various forms, increase blood flow in the clitoris to assist a woman to achieve clitoral engorgement, and otherwise be applicable to the treatment of incontinence and/or the treatment and diagnosis of female sexual disorders. Blood flow is increased by creating a vacuum around and/or using increasing pressure to produce percussion and/or massage of the clitoris, the labia, the external urethral orifice and/or other areas of the female genital region. Pelvic nerve stimulation, such as that caused by suction to and/or engorgement of the clitoris, or suction to the vagina, vaginal wall and/or external urethral orifice, for example, results in clitoral smooth muscle relaxation and arterial smooth muscle dilation via an autonomic spinal reflex arc. This relaxation and dilation result in an increase in clitoral cavernosal artery inflow and an increase in clitoral intracavernous pressure, which lead to tumescence and extrusion of the glans clitoris, according to specific embodiments of the invention.

Moreover, the suction and vibration treatments disclosed in the '643 patent are believed to create pudendal nerve input into the pelvic floor and external sphincter. The pudendal nerve is the primary neurological pathway for the clitoris, both afferent and efferent. As the external sphincter contracts, an impulse is believed sent through the afferent limb of the pelvic nerve, up to the spinal cord at S2, S3 and S4, inhibiting pelvic nerve activity that can contribute to urinary incontinence. In other words, pelvic nerve activity is inhibited by enhancing pudendal nerve activity. With respect to the external sphincter, the efferent aspect is the pudendal nerve, and the afferent aspect is the pelvic nerve. Impulses are sent to the spinal cord, according to embodiments of the invention, where they affect the limb of the pelvic nerve that innervates the bladder.

Related mechanical stimulation techniques are disclosed in U.S. Pat. No. 6,505,630 for treating urinary bladder dysfunction by effective mechanical vibration or stimulation of the external genital area, i.e., the clitoris and/or surrounding external genitalia of women and of the fraenulum praeputii and/or surrounding skin areas of men, including the perineum. It is asserted that such mechanical stimulation is useful for treating urinary bladder dysfunction caused by abnormal urinary detrusor contractions and urethral sphincter dysfunction originating from neurogenic, (e.g. spinal cord injury, scleroses and other neurogenic dysfunctions) as well as non-neurogenic (e.g. stress) causes.

It is assumed that the periodic self-administration or clinical administration of these mechanical stimulation therapies will provide a durable response, i.e., a reduction or elimination of incontinence symptoms that continues for at least a therapeutically significant time period following application of the therapy. It is difficult for the patient to subjectively assess whether the pudendal nerve is necessarily being stimulated during the application of the therapy.

Evoked Response

The delivery of electrical stimulation to or mechanical stimulation of a nerve can cause an evoked response elsewhere in the body. In addition, the delivery of a pacing pulse to heart cells can elicit a responsive cell depolarization and heart contraction if the stimulus energy exceeds a stimulation threshold. It is well known to adjust pacing stimulation energy to a level that exceeds the stimulation threshold sufficiently to ensure reliable pacing while conserving pacing IPG battery energy.

It is also known to assess the evoked response to neural stimulation as described, for example, in U.S. Pat. No. 6,027,456 in the course of positioning spinal cord stimulation electrodes of percutaneous and laminotomy leads within a patient under a general anesthetic. Apparatus disclosed in the '456 patent includes a signal generating device for generating a stimulation signal, where the stimulation signal is delivered to the spinal nerves of the patient via at least two stimulation electrodes of each lead to be implanted, and at least two detection electrodes adapted to be positioned at or about the head of the patient to detect a bodily reaction or evoked response to a stimulation signal from the signal generating device. A feedback device, coupled to the at least two detection electrodes, displays information corresponding to a medial/lateral position of the at least two stimulation electrodes relative to a physiological midline of the patient.

In another embodiment disclosed in the '456 patent, one or more additional detection electrodes are provided which are positioned about the body of the patient to detect a bodily reaction to the stimulation signal from the signal generating device, wherein a position of each additional detection electrode corresponds to a bodily region subject to manageable pain. The additional detection electrodes are also coupled to the feedback device which further displays information corresponding to a longitudinal position of the at least two stimulation electrodes with respect to the dorsal column of the patient. In another embodiment, a patient-specific evoked response model may be created and stored in memory. More specifically, stimulation of various dermatomes or application of electrical energy through implanted stimulating leads (for example, stimulation leads which require revision due to ineffective pain management but remain capable of delivering applied electrical energy) will desirously result in corresponding evoked responses. Prior to or at the time of the procedure, a pattern of evoked potentials may be recorded and evaluated for given input amplitudes, frequencies, pulse widths, or the like. During the subsequent implantation and positioning of stimulating electrodes, evoked potentials may be compared to the previously established evoked potential models at similar amplitudes, frequencies, pulse widths, or the like. An evoked potential model may include the measured data and interpolations between specific measured points to provide an effective means to assess applied stimulation between evaluated lateral positions.

### SUMMARY

The present invention involves the testing of the efficacy of therapeutic mechanical or electrical nerve or pelvic tissue stimulation system particularly for determining the efficacy of such stimulation in evoking a response of pelvic musculature involved in maintaining continence or providing sexual response. In accordance with the present invention, methods are provided to program the implantable pulse generator in a therapy delivery mode to generate and deliver a therapy stimulation regimen comprising electrical stimulation through the medical electrical lead to elicit a contraction of a pelvic muscle to treat at least one of urinary incontinence, fecal incontinence, sexual dysfunction, and pelvic floor weakness.

In preferred embodiments, the testing is automated employing detecting the evoked response to stimulation of a nerve or pelvic muscle tissue employing test stimulation parameters, altering the test stimulation parameters, repeating detecting the evoked response to the altered test stimulation parameters, comparing the evoked responses to determine an optimal or maximal evoked response, and selecting the therapy stimulation parameters as a function of the test stimulation parameters causing the optimal or maximal evoked response.

The objective nature of using evoked potential eliminates the possibility of relying on subjective information from the patient, which may not be suitable for a spinal injury patient or a patient under general or spinal anesthesia or a patient who is suggestible or becomes confused during the test phase, etc. In addition, the record that is established provides an objective measure that physicians and government regulatory bodies may rely on in assessing the potential efficacy of the treatment.

In the context of providing electrical stimulation, the reliance upon the evoked response detected in a test phase reduces the possible complications from infection that would otherwise arise during the prolonged test phase employing a percutaneously implanted neural lead. The battery energy consumed during delivery of therapy stimuli may be minimized by optimally placing the stimulation electrodes with respect to the target nerve, thereby prolonging IPG life or increasing the intervals between recharging of rechargeable batteries powering the IPG. The methods and systems of preferred embodiments of the present invention also advantageously facilitate reprogramming therapy stimulation parameters of therapy stimuli delivered by the IPG in subsequent patient follow-ups. The methods and systems of preferred embodiments of the present invention additionally advantageously facilitates determination that the stimulation electrodes have migrated away from the optimal placement and repositioning of the stimulation electrodes of the neural lead coupled to the IPG.

This summary of the invention has been presented here simply to point out some of the ways that the invention overcomes difficulties presented in the prior art and to distinguish the invention from the prior art and is not intended to operate in any manner as a limitation on the interpretation of claims that are presented initially in the patent application and that are ultimately granted.

### BRIEF DESCRIPTION OF THE DRAWINGS

These and other advantages and features of the present invention will be more readily understood from the following detailed description of the preferred embodiments thereof, when considered in conjunction with the drawings, in which like reference numerals indicate identical structures throughout the several views, and wherein:

FIG. 1 is a schematic view of an exemplary system for positioning neural stimulation electrode(s) and programming or reprogramming a neuromodulation IPG coupled to the neural lead by detecting and assessing the evoked response of a nerve to test stimuli delivered by the IPG to the nerve adjacent the neural stimulation electrode(s) in a test phase;

FIG. 2 is a schematic view of an exemplary system for positioning tissue stimulation electrode(s) and programming a tissue stimulation IPG coupled to the tissue stimulation lead during an initial implantation procedure by detecting and assessing the evoked response of muscle tissue to test stimuli delivered by the IPG to the muscle tissue adjacent the tissue stimulation electrode(s) in a test phase;

FIG. 3 is a flow chart of one method of operating the systems of FIGS. 1 and 2 during initial implantation;

FIG. 4 is a schematic view of an exemplary system for testing the position of tissue stimulation electrodes implanted in urethral sphincter musculature by detecting and assessing the evoked response of muscle tissue to test stimuli delivered by the IPG to the muscle tissue adjacent the tissue stimulation electrode(s) in a test phase;

FIG. 5 is a flow chart of one method of operating the systems of FIGS. 1 and 2 during chronic implantation;

FIG. 6 is a flow chart of a method of positioning tissue stimulation electrodes implanted in urethral sphincter musculature in the first step of the flowchart of FIG. 3 by detecting the sphincter muscle EMG;

FIGS. 7-9 are schematic illustrations of certain of the steps of the flowchart of FIG. 6; and

FIG. 10 is a flow chart of one method of determining the most efficacious mechanical stimulation parameters of a mechanical nerve stimulator applied to a patient's body by detecting and assessing the evoked response of a nerve targeted by the mechanical stimulation in a test phase.

### DETAILED DESCRIPTION OF PREFERRED EMBODIMENTS

The present invention involves the testing of the efficacy of therapeutic mechanical or electrical nerve or muscle tissue stimulation systems particularly for determining the efficacy of such stimulation in evoking a response of the pudendal nerve to mechanical stimulation or the sacral nerve to electrical stimulation or the urinary or anal sphincter musculature in the treatment of various forms of incontinence and sexual dysfunction or other pelvic floor musculature to strengthen it to prevent or alter progression of pelvic floor prolapse. The present invention also involves testing of tissue stimulation lead electrode position in relation to sphincter musculature by monitoring the EMG emanating from the sphincter musculature through use of the tissue stimulation lead or an introducer employed in positioning the lead electrode(s).

Electrical Stimulation of Sacral Nerve

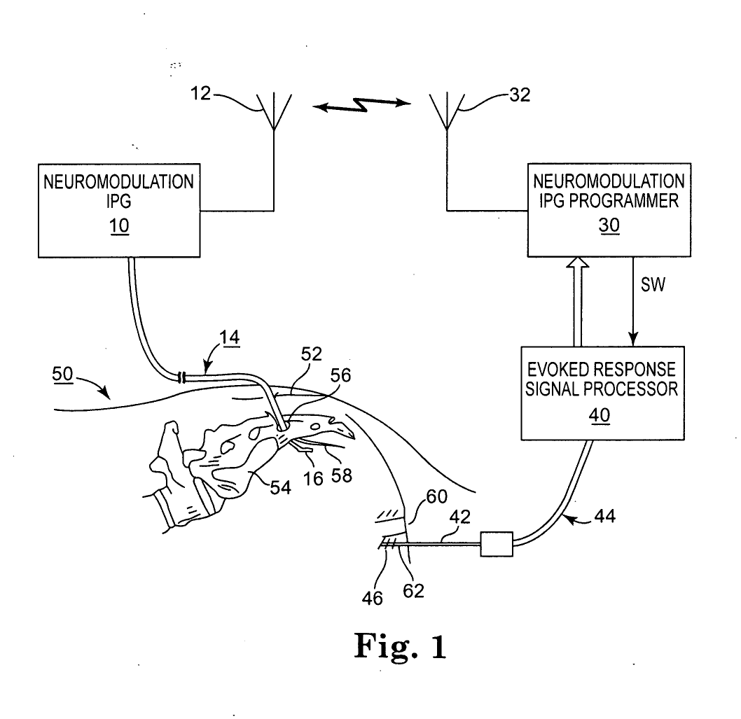

As shown in FIG. 1, methods and apparatus for testing efficacy of therapeutic electrical nerve stimulation of the present invention includes a neurostimulation or neuromodulation IPG 10 that has the ability to switch between a test mode and stimulating or therapy delivery mode, a medical electrical lead, in this case a neural lead, 14 for stimulating a nerve, an IPG programmer 30 for programming the IPG operating modes and test and therapy stimulation parameters, an evoked response sense lead 44, and an evoked response detector or signal processor 40.

In this embodiment, the neural lead 14 is extended through a skin incision 52 of a patient's body 50 and subcutaneously to the sacrum 54 and through a sacral foramen 56 to dispose a distal stimulation electrode(s) 16 adjacent a sacral nerve 58. The electrodes(s) 16 may be a single electrode for unipolar stimulation or two or more electrodes for bipolar or multi-polar stimulation. The neural lead 14 may take any of the known forms and comprises a lead connector at the lead proximal end adapted to be coupled to a connector header of the IPG 10.

The IPG 10 may take any of the known forms that can be programmed to provide therapy stimulation taking the form of single pulses or pulse bursts separated by interpulse periods, wherein the pulse energy, including pulse width and amplitude, and the burst frequency, number of pulses in the burst, and the interpulse period may be remotely programmed by programmer 30. The IPG 10 and lead 12 may provide unipolar or bipolar stimulation of the sacral nerve 58. While the interpulse period may be fixed, delivery of a stimulation therapy may be commanded by programmer 30 or by a limited-function, portable programmer provided to the patient 50 to use to command the IPG 10 to deliver therapy stimuli to stem urge incontinence.

The programmer 30 may take the form of a personal computer having a display, printer, memory, an input device, e.g., a keyboard and mouse or screen pointer, an output coupled to the world-wide web, a CPU, and be controlled by hardware, firmware and software that enables two-way telemetry communication with the IPG 10. The telemetry communication link may take any of the known forms that provide uplink and downlink transmissions between IPG 10 and programmer 20 using antennas 12 and 32 respectively.

The evoked response signal processor 40 comprises a sense amplifier and signal processor that provides an evoked response signal to programmer 30. The evoked response signal processor 40 may be physically incorporated into the programmer 30. The evoked response sense lead 44 is attached to the input of the evoked response signal processor and extends distally to a sense electrode 46 that is placed on the patient's skin or in the patient's body 50 at a point where the evoked response is expected to be transmitted from the stimulated nerve.

It is known that a muscle contracts as a result of control information reaching the muscle from the brain via the nervous system. A nerve impulse, originating in the central nervous system, causes a motor neuron to depolarize a membrane enveloping a small group of muscle fibers that are coupled by an axon to the motor neuron to form a motor unit. The muscle fibers contract sharply, and then relaxes again while other similar motor units are “fired.” A smooth contraction of muscle is a continuous cyclic process of many motor units firing and relaxing and is evidenced by the EMG. The urinary and anal sphincter musculature and other pelvic floor muscles comprise such motor units, which are constantly in a state of active contraction, except during the voluntary act of evacuation, to maintain normal bladder control. This muscular contraction supports the pelvic and abdominal contents, and this maintains a constant closure of the urethral and anal orifices. The contraction effects elevation of the normal bladder neck sufficiently to ensure that it remains closed. Electrical stimulation to nerves innervating sphincter and pelvic floor musculature or directly to such musculature may enhance the strength of the contractions.

The evoked response may comprise an EMG of a pelvic floor muscle or sphincter that is triggered to constrict by the nerve that is activated by the applied stimulation, the sacral or pudendal nerve in the example depicted in FIG. 1. The application of electrical stimulation to the nerves may cause an evoked response that comprises an increase in the amplitude or a characteristic activity pattern of the EMG during the application of the electrical stimulation. The evoked response may be detected employing a variety of sense electrodes, including electrodes applied to the patient's scalp.

In this embodiment depicted in FIG. 1, the sense electrode 46 is preferably a small diameter needle electrode at the tip of a needle 42 that is inserted through the skin and into the internal or external anal sphincter 62 for the duration of the testing phase to detect the EMG during the test window. The successful stimulation of the sacral nerve 58 is expected to elicit an evoked response in the muscle cells of the anal sphincter 62 surrounding anus 60. It may also be possible to substitute skin EMG electrodes against the skin over the sphincter 62 in substitution for the needle electrode.

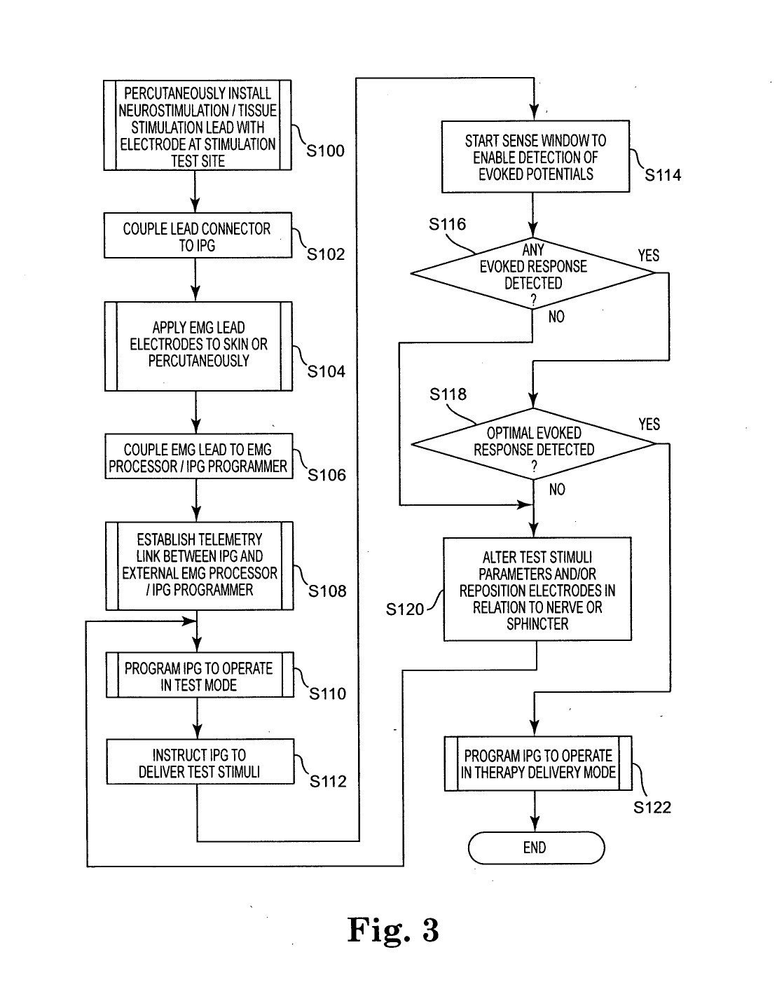

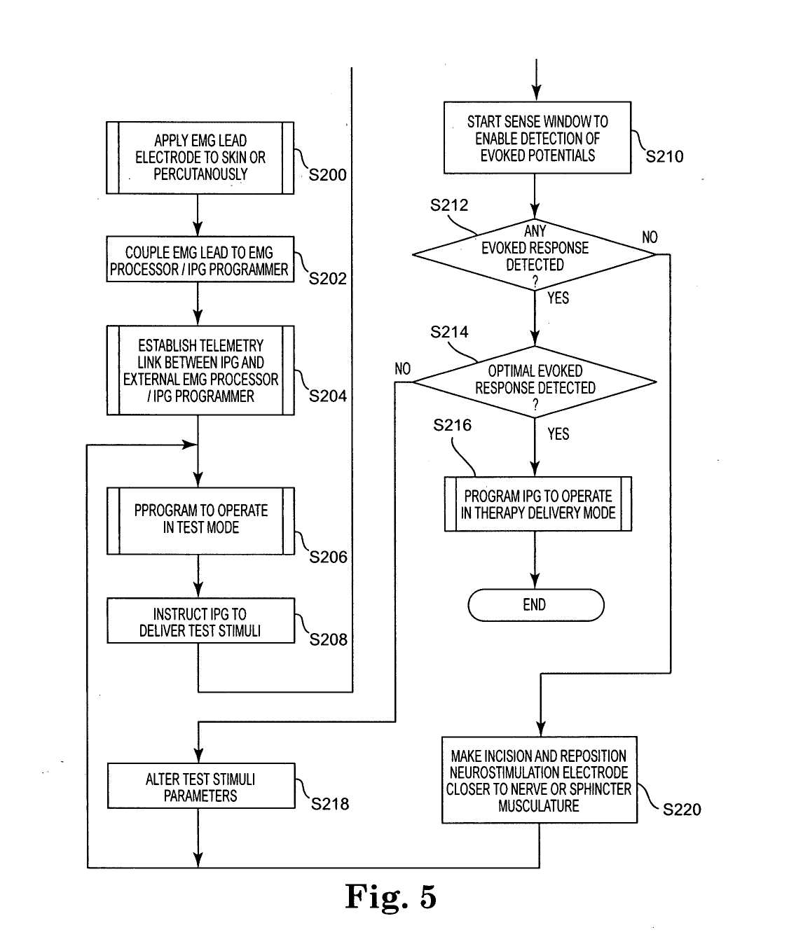

In accordance with the present invention, the IPG 10 is capable of operating in a test mode to perform the test phase and in a therapy mode to deliver the programmed therapy stimuli to the sacral nerve 58. The neuromodulation IPG programmer 30 is similarly capable of effecting such programming of the operating modes and stimulation parameters of the IPG 10. The testing steps undertaken during the implantation of the neural lead 14 and during chronic implantation of the neuromodulation IPG 10 and neural lead 14 are depicted in FIGS. 3 and 5, respectively.

Electrical Stimulation of Urethral Sphincter and/or Pelvic Floor Muscles

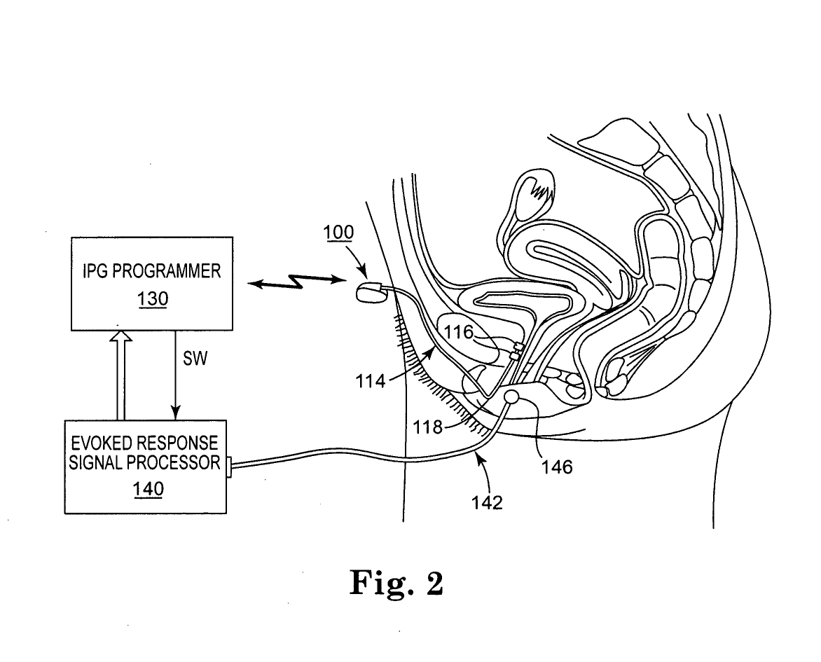

As shown in FIG. 2, methods and apparatus for testing efficacy of therapeutic electrical nerve stimulation of the present invention include a tissue stimulation IPG 100 similar to neuromodulation IPG 10 that has the ability to switch between a test mode and stimulating mode, a medial electrical lead, in this case a tissue stimulation lead, 114 for stimulating the urinary sphincter musculature, an IPG programmer 130 for programming the IPG operating modes and test and therapy stimulation parameters, an evoked response sense lead 142, and an evoked response detector or signal processor 140. The tissue stimulation IPG 100 and tissue stimulation lead 114 may take the forms disclosed in the above-referenced Biocontrol Medical patents. The electrodes(s) 116 may be a single electrode for unipolar stimulation or two or more electrodes for bipolar or multi-polar stimulation. The tissue stimulation lead 114 may take any of the known forms and comprises a lead connector at the lead proximal end adapted to be coupled to a connector header of the IPG 100. In this example, a bipolar tissue stimulation lead 114 having a pair of stimulation electrodes 116 disposed in the urethral sphincter musculature is depicted. The tissue stimulation lead 114 is operable, when coupled to the IPG 100, to transmit EMG signals to the IPG sense amplifier (if present in the IPG), and to deliver the stimulation from an IPG output circuit to a stimulation site of the patient's body, particularly the region of the urethra, in the treatment of incontinence.

The IPG programmer 130 may take the form of the above-described IPG programmer 30 with device specific software enabling uplink and downlink telemetry communication with the IPG 100. The evoked response detector or signal processor 140 and the evoked response sense lead 142 may take the form of the above-described evoked response detector or signal processor 40 and the evoked response sense lead 42. The evoked response detector or signal processor 140 may be incorporated in or combined with the IPG programmer 130. In this example, the evoked response sense lead 142 terminates in is a skin surface contact electrode 146 adapted to be disposed against the patients skin or within the urethra or the vagina (in the case of a female patient}.

Again, the evoked response may comprise an increased amplitude or a characteristic pattern in the EMG of a pelvic floor muscle or the sphincter that is triggered to constrict by the applied stimulation, the urethral sphincter musculature in this example.

Certain implantation methods for implanting the tissue stimulation IPG 100 and tissue stimulation lead 114 in the body of a female patient are described in the above-referenced Biocontrol Medical '651 and '480 patents. It is suggested that similar methods would be employed in the implantation of the tissue stimulation IMD in a male patient and in positioning the stimulation electrodes 116 in relation to male or female anal sphincter musculature to apply therapeutic stimulation to alleviate fecal incontinence.

In one implantation method shown in FIGS. 2A-2G of the '480 patent, a skin incision is made at a labial site approximately 0.5-1 cm anterior and lateral to the urethral meatus. A 5 French, splittable short introducer is inserted into the skin incision adjacent to the lead and advanced with care slightly medially, i.e., towards the urethra, about 2.5 cm, to a site 0.5-1 cm lateral to the urethral wall. The electrode and fixation mechanism (a spiral helix or hook) are advanced through the splittable introducer lumen of the introducer extending from the skin incision to the stimulation and fixation site proximate the urethral sphincter. The introducer sleeve is split apart to withdraw it over the lead body after the stimulation electrode is properly positioned. The stimulation lead body is sutured to the subcutaneous tissue to secure it from movement.

A subcutaneous tunnel or pathway is tunneled between the pocket and the skin incision, and the lead body is extended through the pathway to dispose a distal portion of the lead outside the skin incision. In one embodiment of the '480 patent, the tunneling of the lead body between the skin incision and the suprapubic incision is effected by subcutaneously tunneling a 12 Fr introducer from the either incision to the other incision and passing the lead, distal end first, from the suprapubic incision through the introducer lumen to the skin incision and then removing the introducer over the lead body. The exposed distal portion of the lead body is retracted subcutaneously, and the skin incision is closed.

In the step in the testing of position of the electrodes 116 illustrated in FIG. 2, the tissue stimulation IPG 100 is disposed outside a subcutaneous pocket formed to receive the IPG within the patient's body and is coupled to the proximal lead connector in a manner well known in the art. The body of the tissue stimulation lead 114 extends subcutaneously and proximally from a skin incision to the tissue stimulation IPG 100 and distally alongside the urethra to the distal stimulation electrodes 116. The segment of the lead body 118 exposed from the skin incision can be grasped to push or pull the distally extending segment of the lead body to adjust the position of the distal stimulation electrodes 11 6.

Electrode(s) Positioninci Durinci Initial Implantation

FIG. 3 illustrates one method employing the apparatus of either FIG. 1 or FIG. 2 of initially placing the stimulation and sense electrodes 16 or 116 in steps S100-S106, conducting the testing steps S108-S120 in the test phase, and programming the therapy parameters of the therapy stimuli in step S122. It will be understood that the implantation procedure may electively be terminated if an evoked response cannot be detected in step S116 after a number of failed attempts. For convenience, the steps of the flow charts of FIGS. 3 and 5 refer to the evoked response sense lead 44 or 144 as an EMG lead, the evoked response signal processor 40 or 140 as an EMG processor, and the sense electrode 46 or 146 as an EMG lead electrode.

In the test mode of the neurostimulation IMD depicted in FIG. 1, the neural lead electrode(s) 16 and the sense electrode(s) 46 are placed as shown in FIG. 1 following steps S100-S104. Similarly, in the test mode of the tissue stimulation IMD depicted in FIG. 2, the tissue stimulation electrodes 116 and the sense electrode 146 are placed as shown following steps S100-S104. The EMG lead 44 or 144 is coupled to the respective evoked response detector or EMG processor 40 or 140 in step S106.

In step S108, the IPG programmer 30 or 130 is operated establish a telemetry link with the IPG 10 or 100, respectively. In step S110, the user selects a test stimulation regimen and stimulus parameters and causes the programmer 30 or 130 to downlink telemetry transmit the selected test regimen and a mode change command to operate the IPG 10 or 100 in the test mode.

In step S112, the programmer 30 or 130 generates a command that is downlink telemetry transmitted to the IPG 10 or 100 to instruct the IPG 10 or 100 to deliver the test stimuli with the specified test stimulation parameters. The IPG 10 or 100 may uplink telemetry transmit a confirmation of delivery of the test stimuli. In step S114, the programmer 30 or 130 may initiate timeout of a sense window SW starting at or prior to the delivery of the test stimuli and continuing for a time following termination of the test stimuli delivery to enable. The sense window may be displayed on the programmer screen in relation to the display of the EMG and may be used to enable the evoked response detector 40 or 140 to detect any evoked response in the EMG during the sense window SW. Or, step S114 of timing out a sense window SW may not be included in the test method of FIG. 3.

As noted above, the evoked response may comprises a change in the EMG generated in the patient's body during the sense window SW that signifies that the test stimuli delivered in the vicinity of the nerve or muscle intended to be stimulated has in fact stimulated the nerve or muscle tissue. It will be understood that the evoked response may itself constitute or reflect particular characteristics of the delivered test stimuli conducted through the body. Furthermore, the test stimulation regimens may include therapy regimens to enable the user to select the optimal therapy regimen or test stimuli that are not part of a range of therapy regimens but simply are employed to position the electrodes 16 or 116.

Thus, the test stimulation parameters, principally the pulse amplitude, pulse width, and frequency and the number of pulses 1-N of a burst of pulses, of the test stimuli may differ from the stimulation parameters of the therapy stimuli. The therapy stimulation pulses may also be delivered across bipolar electrodes 16 or 116 in the therapy delivery mode, whereas the test stimulation pulses may be delivered in a unipolar mode between one stimulation electrode 16 or 116 and the IPG housing acting as an indifferent electrode. The test stimulation parameters employed in the test phase to determine an optimal evoked response may be more battery energy draining than is necessary to provide a therapy. In other words, the steps undertaken during the test phase or mode may require relatively high-energy test stimuli facilitate provoking the evoked response and optimally placing the stimulation electrodes with respect to the nerve. Lower energy therapy stimuli may be sufficient to therapeutically lessen incontinence severity or events.

In steps S116 and S118, the detection or failure to detect an evoked response or an optimal evoked response following delivery of each test stimuli is evaluated. The waveform and peak amplitude of the EMG detected and displayed during delivery of each test stimulation regimen can be observed by the user, and the user may identify the optimal evoked response associated with a particular test stimulation regimen. Alternatively, the waveform and peak amplitude or other signal characteristics of each the EMG detected and displayed during delivery of each test stimulation regimen can be processed and stored in memory. Comparison logic may be incorporated in the IPG programmer 30 or 130 to identify the optimal evoked response from the waveform and peak amplitude or other signal characteristics of each the EMG and to associate the test stimulation regimen with it.

In step S120, the test stimulation parameters may be altered and/or the stimulation electrodes 16 or 116 may be repositioned for continued testing starting at step S110. The evoked response signal may be measured in amplitude and displayed on the programmer screen to determine any evoked response in step S116 and an optimal evoked response in step S118. The implantation procedure may be terminated if it is not possible to elicit any evoked response in step S116 or if an evoked response requires test stimulation parameters that are unrealistically high. The steps of the present invention can also be accompanied by interviewing the patient to correlate the patient's subjective response to the displayed evoked response.

The therapy stimulation parameters and the test stimulation parameters may be correlated in memory in the programmer 30 or 130 or in memory in IPG 10 or 100 so that the therapy stimulation parameters may be programmed in step S122 as a function of the optimal evoked response detected in step S118. Alternatively, the user enters the therapy stimulation parameters and reset the IPG to the therapy delivery mode in step S122. A patient test record is created and stored in memory for potential future use during subsequent patient follow-up and in reprogramming the stimulation parameters of the therapy stimuli generated by the IPG 10 or 100.

Evoked Response Testinci and Electrode(s) Repositioninci During Chronic Implantation

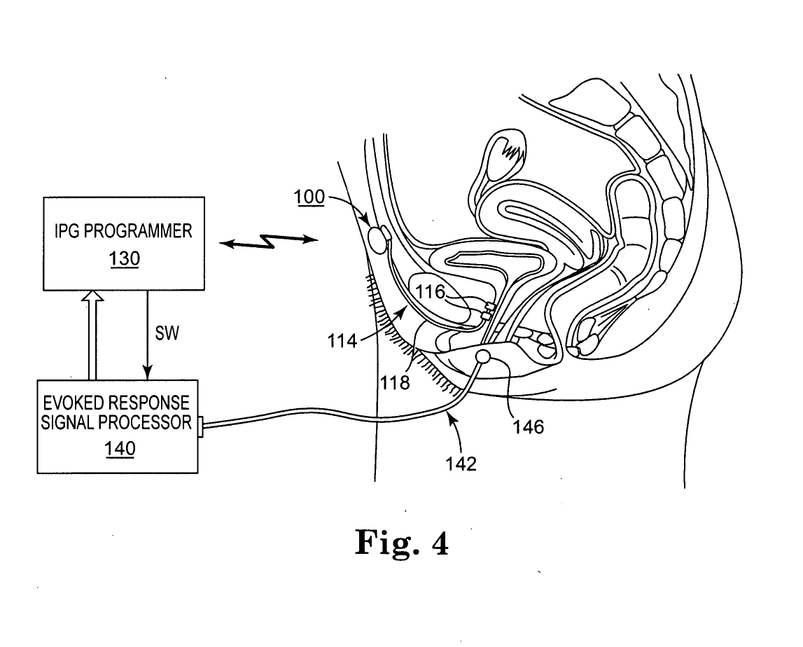

FIG. 5 illustrates one method employing the apparatus of either FIG. 1 or FIG. 2 of periodically testing the position of the electrode(s) 16 or 116 and the response of the nerve or tissue to the programmed therapy stimulation regimen. For example, FIG. 4 illustrates the tissue stimulation IPG 100 disposed within the subcutaneous pocket formed to receive the IPG 100 within the patient's body and tissue stimulation lead 114 extending subcutaneously and distally alongside the urethra to the distal stimulation electrodes 116. If necessary to reposition the lead electrodes 116, a segment of the lead body may again be exposed by making a skin incision to grasp the lead body 118 to push or pull the distally extending segment of the lead body 118 to adjust the position of the distal stimulation electrodes 116.

Again, the EMG lead 44 or 144 is coupled to the respective evoked response detector 40 or 140 and the patient's skin in steps S200 and S202. The telemetry link between the IPG 10 and the IPG programmer 30 or the IPG 100 and the IPG programmer 130 is established in step S204, and the IPG 10 or 100 is programmed to operate in the test mode in step S206. The user operates the IPG programmer 30 or 130 to enter the test mode and to select the test stimulation parameters as described above with respect to step S110 of FIG. 3.

In step S208, the programmer 30 or 130 generates a command that is downlink telemetry transmitted to the IPG 10 or 100 to instruct the IPG 10 or 100 to deliver the test stimuli with specified test stimulation parameters entered in step S206. The sense window is started in step S210 in any of the manners described above in regard to step S114 of FIG. 3. As noted above, the evoked response may comprise a change in the EMG amplitude or signal pattern generated in the patient's body during the sense window SW that signifies that the test stimuli delivered in the vicinity of the nerve or muscle intended to be stimulated has in fact stimulated the urethral sphincter or other pelvic floor muscle tissue.

Steps S212, S214 and S216 are followed in the same manner as steps S116, S118, and S122 of FIG. 3 described above. Step S218 may comprise part of step S120 of FIG. 3 described above. If an evoked response cannot be detected or is insufficiently low in amplitude, then the physician may resort to making an incision to expose a segment of the lead body and reposition the electrodes 16 or 116. A suitable skin incision is depicted in FIG. 2 to expose the lead body 1118 of tissue stimulation lead 114 to enable repositioning by retracting or advancing the distal segment of the lead body 118 within the urethral sphincter musculature. Then, steps S206-S218 are repeated for each change in electrode position.

EMG Detection

In accordance with this aspect of the present invention, the EMG is sensed by an electrode placed within the urethral sphincter alongside the urethral axis on or close to the musculature to aid in positioning the tissue stimulation electrode(s), e.g., tissue stimulation electrodes 116. The EMG emanating from the muscle indicates the activity of that urethral sphincter or other pelvic floor muscles attempting to maintain bladder control without any applied electrical stimulation.

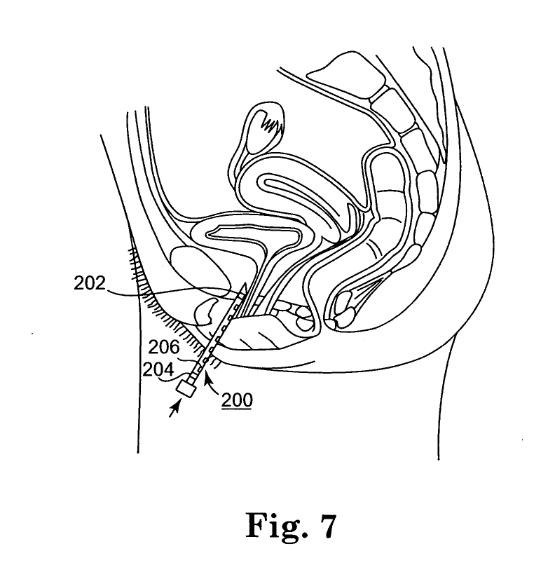

In accordance with one embodiment of this aspect of the present invention, a lead introducer 200 depicted in FIGS. 7-9 is provided to detect the EMG and aid in positioning the lead electrodes 116 during the initial implantation step S100 of the flowchart of FIG. 3. The lead introducer may be splittable along its length.

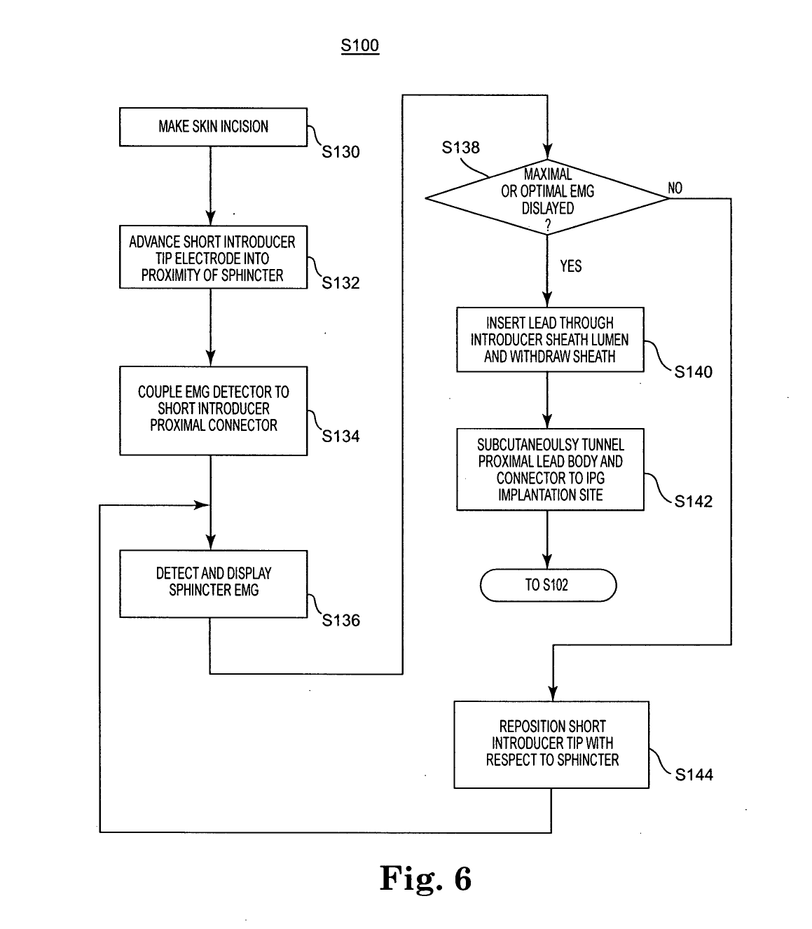

The lead introducer 200 of the type described above is modified to have a nonconductive introducer sheath that bears an exposed EMG sense electrode 202 at the sheath distal end that is coupled by an insulated conductor 206 to a connector or exposed connection surface 204 near the sheath proximal end. The steps depicted in FIG. 6 are employed using the introducer 200 and an EMG signal detector and display 60, which may be incorporated into the IPG programmer 130 as step S100 in FIG. 3

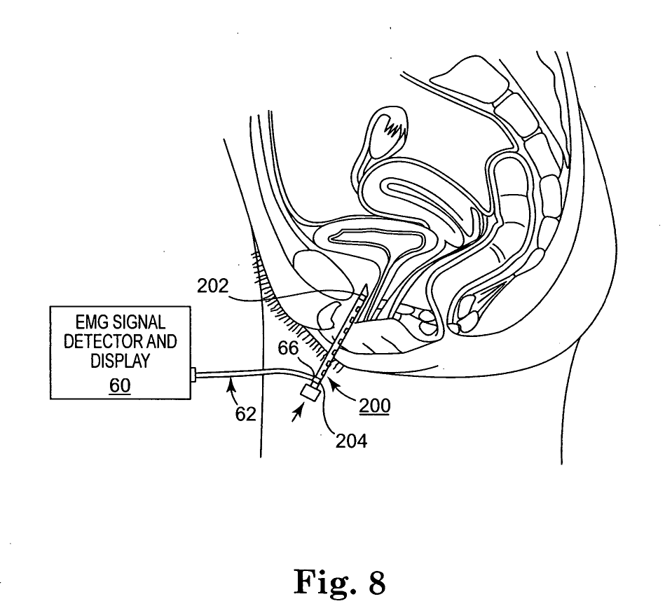

Thus, in steps S130 and S132, the skin incision is made and the tip of the short introducer 200 is advanced alongside the urethra as shown in FIG. 7. In FIG. 8 illustrating step S134, an EMG signal cable 62 is coupled to the EMG detector and display 60 and is attached by a lead connector 66 to the sheath connector or connection surface 204. In step S136, the EMG is detected and displayed as the distal tip of introducer 200 is advanced from the skin incision in relation to the urethral tissue. The optimal or maximal EMG is detected by following and repeating steps S138, S144, and S136. The site of the introducer electrode 202 providing a maximal EMG is selected as the stimulation site for positioning the tissue stimulation electrodes 116.

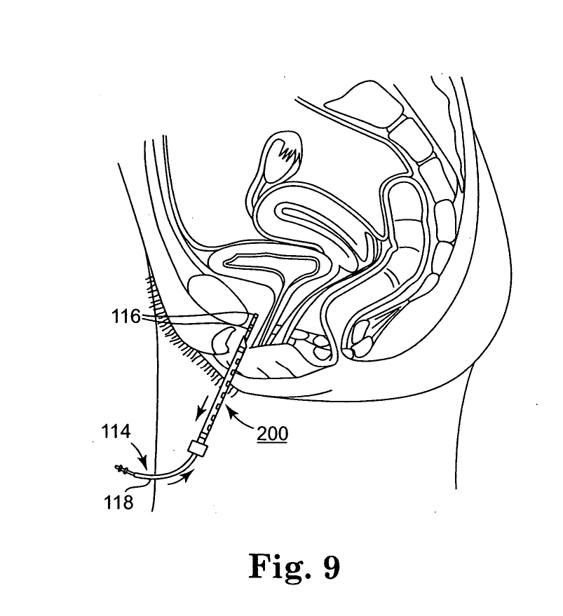

After the optimal stimulation site is determined, the introducer 200 is retracted proximally a distance related to the length of the tissue stimulation electrodes 116. In step S140 illustrated fin FIG. 9, the tissue stimulation lead 114 is advanced distally through the introducer lumen to dispose the tissue stimulation electrodes 116 at the selected site of implantation. The lead introducer 200 may be split away, if splittable, or withdrawn over the lead connector as the lead body 118 is held in position distal to the introducer 200, so that the tissue stimulation electrodes 116 are not dislodged from the stimulation site. It will be understood that the EMG signal detector and display 60 may be coupled to the lead connector elements to detect the EMG using the tissue stimulation electrodes 116 after the lead introducer 200 is removed.

In step S142, the subcutaneous pathway from the skin incision to the IPG implantation site is created using a lead tunneler, and the proximal segment of the lead body 118 is advanced through the pathway to dispose the lead connector at the IPG implantation site or pocket for connection to the IPG 100 as shown in FIG. 2. Then, steps S102-S122 may be followed as described above.

Mechanical Nerve Stimulation

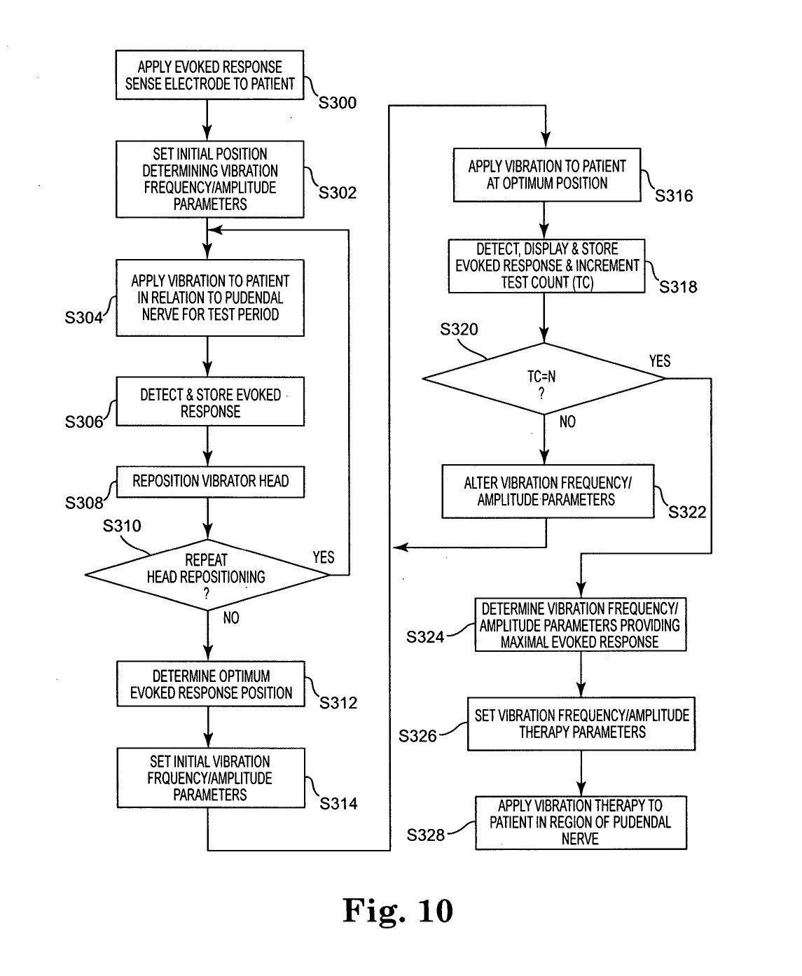

Similar steps of determining the evoked response to applied mechanical stimulation of the patient's body overlying a nerve, e.g., the pudendal nerve or its sensor receptors, with a vibrator that is adjustable in mechanical amplitude and frequency are set forth in FIG. 10. The system employs an evoked response detector or signal processor, a sense lead for disposing a sense electrode adjacent to or in tissue where an evoked response to the applied mechanical stimulation would be expected, and a feedback system for automatically adjusting the mechanical stimulation parameters following the steps of FIG. 10

The preferred embodiment involves mechanical stimulation of the pudendal nerve (through its sensor receptors) to treat incontinence. In step S300, the evoked response sense electrode is applied on or in the patient's body, e.g., at the anal canal, lower urinary tract, near the pudendal nerve, sacrum, or spine and in or on the scalp. The initial or starting vibration amplitude and frequency for locating the optimal vibration head location is set in step S302, and the vibration head of the vibrator is applied in step S304 to the perineal skin area over the pudendal nerve.

In steps S306-S312, the optimum position of the vibrator head on the patient's body is determined as a function of the maximal evoked response that is detected. Then, the stimulation parameters are automatically altered and applied “N” times as N evoked response magnitudes are detected and stored in steps S314-S320. The stimulation parameters that effect the maximal evoked response are determined in step S322 and employed in steps S324 and S326 in the therapy session.

The mechanical vibration or stimulation can for example be performed by a vibrator source as known from PCT Patent Application No. WO 96/32916. A physician, physiotherapist, nurse or the like can operate the vibrator to conduct the optimization steps of FIG. 3 in a clinical setting. Then, the patient can be provided with the vibrator with the vibration parameters set in step S326 and personally operate it according to a prescribed schedule over a period of months to reduce incontinence. As set forth in the above-referenced '630 patent, external vibratory stimulation may be performed daily (or with days interval) for periods of 0.1 to 5 minutes, typically 3 minutes, and the maximum numbers of stimulation periods may be 6 sessions per day, for a daily total stimulation of up to 30 minutes.

Although the electrical and mechanical stimulation treatments described above relate to alleviating incontinence, it will be understood that they may find application in the treatment of sexually dysfunctions.

All patents and publications referenced herein are hereby incorporated by reference in their entireties.

It will be understood that certain of the above-described structures, functions and operations of the above-described preferred embodiments are not necessary to practice the present invention and are included in the description simply for completeness of an exemplary embodiment or embodiments. It will also be understood that there may be other structures, functions and operations ancillary to the typical surgical procedures that are not disclosed and are not necessary to the practice of the present invention.

In addition, it will be understood that specifically described structures, functions and operations set forth in the above-referenced patents can be practiced in conjunction with the present invention, but they are not essential to its practice.

It is therefore to be understood, that within the scope of the appended claims, the invention may be practiced otherwise than as specifically described without actually departing from the spirit and scope of the present invention.

1. A system of testing the efficacy of therapeutic stimulation applied to a patient by a therapy delivery device to alleviate one of incontinence or sexual dysfunction comprising:
an evoked response detector having an evoked response sense electrode adapted to be applied to a patient's body, the evoked response detector adapted to display an evoked response to stimulation applied to the patient's body; and
a therapy delivery device operable in a therapy delivery mode to deliver a therapy stimulation regimen to a stimulation site of a patient's body and a test mode for delivering test stimulation regimens to the stimulation site, the therapy delivery device further comprising means operable in the test mode for formulating series of test stimulation regimens and applying each test stimulation regimen to the stimulation site while the evoked response detector detects any evoked response to each test stimulation regimen,
whereby a test stimulation regimen that causes an optimal evoked response may be determined.

2. The system of claim 1, wherein the therapy delivery device is programmable to deliver a therapy stimulation regimen associated with the test stimulation regimen determined to cause the optimal evoked response in the therapy delivery mode.

3. The system of claim 1, wherein the therapy delivery device comprises an implantable pulse generator and a medical electrical lead having a stimulation electrode adapted to be positioned in the patient's body in operative relation to a pelvic nerve or musculature.

4. The system of claim 3, wherein the implantable pulse generator is programmable in a therapy delivery mode to generate and deliver a therapy stimulation regimen comprising electrical stimulation through the medical electrical lead to elicit a contraction of a pelvic muscle to treat at least one of urinary incontinence, fecal incontinence, sexual dysfunction, and pelvic floor weakness.

5. The system of claim 4, wherein the implantable pulse generator is programmable in a test mode to generate and deliver a test stimulation regimen comprising electrical stimulation through the medical electrical lead to elicit a contraction of a pelvic muscle that manifests an evoked response.

6. The system of claim 5, wherein the therapy delivery device is programmable to deliver a therapy stimulation regimen associated with the test stimulation regimen determined to cause the optimal evoked response in the therapy delivery mode.

7. The system of claim 3, wherein the therapy delivery device is programmable to deliver a therapy stimulation regimen associated with the test stimulation regimen determined to cause the optimal evoked response in the therapy delivery mode.

8. The system of claim 1, wherein the therapeutic and test stimulation comprises mechanical stimulation that evokes contraction of a pelvic muscle to treat at least one of urinary incontinence, fecal incontinence, sexual dysfunction, and pelvic floor weakness.

9. A method of testing the efficacy of therapeutic stimulation applied to a patient by a therapy delivery device to alleviate one of incontinence or sexual dysfunction comprising:
operating the therapy delivery device in a test mode, and:
(a) applying an evoked response sense electrode to the patient's body and coupling the evoked response sense electrode to an evoked response detector;
(b) applying a test stimulation regimen to body tissues at a stimulation site;
(c) operating the evoked response detector to detect any evoked response to the applied test stimulation regimen;
(d) adjusting one or both of test stimulation parameters and the stimulation site and repeating steps (c) and (d); and
(e) identifying a maximal evoked response and an optimal therapy stimulation regimen; and
operating the therapy delivery device in a therapy delivery mode to deliver the optimal therapy stimulation regimen to the patient's body.

10. The method of claim 9, wherein the therapeutic and test stimulation comprises electrical stimulation.

11. The method of claim 9, wherein the therapeutic and test stimulation comprises electrical stimulation applied to pelvic musculature to elicit a contraction of a pelvic muscle to treat at least one of urinary incontinence, fecal incontinence, sexual dysfunction, and pelvic floor weakness.

12. The method of claim 11, wherein the pelvic muscle includes one of a urinary and an anal sphincter.

13. The method of claim 9, wherein the therapeutic and test stimulation comprises electrical stimulation applied to a nerve to elicit a contraction of a pelvic muscle to treat at least one of urinary incontinence, fecal incontinence and sexual dysfunction.

14. The method of claim 13, wherein the pelvic muscle includes one of a urinary and an anal sphincter.

15. The method of claim 9, wherein the therapeutic and test stimulation comprises mechanical stimulation that evokes sensory afferent pulses or contraction of a pelvic muscle to treat at least one of urinary incontinence, fecal incontinence, sexual dysfunction, and pelvic floor weakness.

16. The method of claim 15, wherein the pelvic muscle includes one of a urinary and an anal sphincter.

17. A method of determining the efficacy of electrical stimulation of sphincter musculature of the pelvic floor that spontaneously exhibits an EMG to control incontinence comprising:
inserting an EMG sense electrode into relation with the sphincter musculature;
detecting the EMG of the sphincter musculature;
determining a stimulation site of a tissue stimulation electrode of a medical electrical lead from the detected EMG;
implanting the medical electrical lead by positioning the stimulation electrode at the stimulation site;
coupling the medical electrical lead to an implantable pulse generator capable of operating in a programmed therapy delivery mode and a test mode;
disposing an evoked response sense electrode at a sense site of the patient's body;
operating the implantable pulse generator in the test mode applying a test stimulation regimen through the tissue stimulation electrode to the stimulation site;
detecting at the sense electrode an evoked response to test stimuli of the test stimulation regimen;
determining optimal therapy stimulation parameters from one or more detected evoked response; and
adjusting a parameter of the programmed therapy stimulation mode to reflect the optimal therapy stimulation parameters.

18. The method of claim 17, wherein the determining step comprises:
formulating a series of test stimulation regimens;
repeating the steps of operating the implantable pulse generator in the test mode applying each test stimulation regimen through the tissue stimulation electrode to the stimulation site and detecting at the sense electrode an evoked response to test stimuli of each test stimulation regimen;
comparing the evoked responses to ascertain the optimal evoked response; and
associating the optimal evoked response with a therapy stimulation regimen exhibiting the optimal therapy delivery parameters.

19. The method of claim 17, wherein the therapeutic and test stimulation comprises electrical stimulation applied to pelvic musculature to elicit a contraction of a pelvic muscle to treat at least one of urinary incontinence, fecal incontinence, sexual dysfunction, and pelvic floor weakness.

20. The method of claim 19, wherein the pelvic muscle includes one of a urinary and an anal sphincter.
